(19) United States
(12) Patent Application Publication
(10) Pub. No.: US 2018/0265829 A1
(43) Pub. Date: Sep. 20, 2018

(54) KIT FOR ISOLATING ADIPOSE TISSUE-DERIVED STROMAL VASCULAR FRACTION AND METHOD FOR ISOLATING ADIPOSE TISSUE-DERIVED STROMAL VASCULAR FRACTION USING SAME

(71) Applicants: Dae Yong KIM, (US); NBIOTEK, INC, Gyeonggi-do (KR)

(72) Inventors: Dae Yong KIM, Gyeonggi-do (KR); Min Ah YANG, Gyeonggi-do (KR)

(21) Appl. No.: 15/762,301

(22) PCT Filed: Oct. 16, 2015
(86) PCT No.: PCT/KR2015/010974

§ 371 Date: Mar. 22, 2018

(30) Foreign Application Priority Data
Sep. 25, 2015 (KR) 10-2015-0136971

(51) Int. Cl. C12M 1/12; C12N 5/0775

(52) U.S. Cl. CPC C12M 1/12; A61K 35/35; C12N 5/0667

(57) ABSTRACT

A kit for separating adipose tissue-derived stromal vascular fraction is easy to operate so that even a non-specialist can use the kit and can perform washing and suspending processes for stromal vascular fraction in one tube repeatedly at the same time, thereby minimizing exposure to air, resulting in a low risk of secondary infection caused by external agents. Accordingly, a survival rate and a recovery rate of the stromal vascular fraction with respect to the amount of adipose tissue are increased whereby less adipose tissue is required and thus the stromal vascular fraction is efficiently and easily separated from the adipose tissue.

### TECHNICAL FIELD

The present invention relates to a kit for separating adipose tissue-derived stromal vascular fraction and a method of separating adipose tissue-derived stromal vascular fraction using same. More particularly, the present invention relates to a kit for separating adipose tissue-derived stromal vascular fraction and a method of separating adipose tissue-derived stromal vascular fraction using same, in which the stromal vascular fraction is easily separated from the adipose tissue by using a novel structure of the present invention without expensive equipment.

### BACKGROUND ART

Stromal vascular fraction (SVF) extracted from adipose tissue includes adipose stem cells, fibroblasts, immune cells, vascular endothelial cells, and so on, and those cells secrete extracellular matrix proteins such as various growth factors, collagen, and hyaluronic acid. Therefore, when the stromal vascular fraction is injected into skin, hyaluronic acid secretion increases whereby a skin-moisturizing effect increases, and elastic proteins such as collagen, elastin, etc., becomes rich whereby firmness of skin increases. Accordingly, demands for a cosmetic procedure using the stromal vascular fraction have increased rapidly in recent years due to such effects.

However, because it is tricky to extract pure stromal vascular fraction from adipose tissue, equipment with high cost is required, so cost for a cosmetic procedure using the stromal vascular fraction is expensive. Accordingly, a kit for separating the stromal vascular fraction easily becomes urgently necessary, and Korean Patent Publication No. 2015-0051026 discloses a tube for mass isolation of adipose stem cells (stromal vascular fraction). However, according to the related art, when reagents for separating adipose tissue-derived stromal vascular fraction are mixed, there is a high possibility that the stromal vascular fraction leaks through a mesh of a first chamber, so a recovery rate is low. In addition, a specific layer generated by repeated centrifugations is required to keep removed, and such process is cumbersome and complex for non-specialists to perform.

Therefore, a kit for separating adipose tissue-derived stromal vascular fraction with a high recovery rate and an easy operation is required to be developed.

### DISCLOSURE

### Technical Problem

Accordingly, the present invention has been made keeping in mind the above problems occurring in the related art, and thus the inventors of the present invention developed a kit realizing high recovery rate and survival rate of the stromal vascular fraction and easy operation, thereby completing the present invention.

An object of the present invention is to provide a kit for separating adipose tissue-derived stromal vascular fraction, the kit including a first separation tube separating the stromal vascular fraction from adipose tissue and a second separation tube washing and suspending the stromal vascular fraction separated in the first separation tube.

In addition, another object of the present invention is to provide a method of separating the stromal vascular fraction from the adipose tissue efficiently by using the kit.

### Technical Solution

In order to accomplish the above object, a method according to the present invention includes:

(a) a first separation tube for separating stromal vascular fraction from adipose tissue, the first separation tube including an upper storage portion, a lower storage portion tapering downwards, a filter separating the upper storage portion and the lower storage portion from each other and allowing the stromal vascular fraction to pass therethrough but not the adipose tissue, at least one opening portion for introducing/discharging a material and air, a cap closing the opening portion, a bottom hole disposed at a lower end of the lower storage portion, and a bottom hole blocking film disposed at a lower end of the bottom hole to protect the bottom hole from outside and composed of a material penetrated by an injection needle or a syringe; and

(b) a second separation tube for washing and suspending the stromal vascular fraction, the second separation tube including an upper storage portion tapering downwards and provided with a spiral at an upper end thereof, a lower storage portion tapering upwards and downwards, a passage portion connecting between the upper storage portion and the lower storage portion, at least one opening portion for introducing/discharging a material and air, a cap closing the opening portion, a closing adjustment screw capable of moving vertically, closing the passage portion, and engaging with the spiral provided on an upper end of the upper storage portion, a bottom hole disposed at a lower end of the lower storage portion, and a bottom hole blocking film composed of a material penetrated by an injection needle or a syringe, disposed at a lower end of the bottom hole to protect the bottom hole from outside.

A head portion of the closing adjustment screw engaged with the spiral provided on the upper end of the upper storage portion of the second separation tube may be a cap for the upper storage portion.

The upper storage portion of the second separation tube may further include a projection at an upper end thereof, and the spiral where the closing adjustment screw engages is formed inside or outside of the projection.

A silicone cap may be provided at an end of the closing adjustment screw of the second separation tube to close the passage portion tightly.

The passage portion of the second separation tube may have a scale to adjust a volume of contents in the lower storage portion.

The closing adjustment screw of the second separation tube may be provided with a pressure control ring at an end thereof, the pressure control ring including a plurality of holes. In addition, the pressure control ring may be provided with a blocking plate at an upper end thereof or a blocking plate at each of an upper end and a lower end thereof, the blocking plate opening and closing the plurality of holes. An upper blocking plate and a lower blocking plate may open and close different holes when the blocking plates are provided on the upper and lower ends of the pressure control ring.

Each bottom hole blocking film of the first separation tube and the second separation tube may be provided with a groove at a lower end surface, the groove indicating a position of the bottom hole disposed at a lower end of the lower storage.

Each of the first separation tube and the second separation tube may further include a sealing membrane at a lower portion of the bottom of the blocking film, and the sealing membrane is composed of a material penetrated by an injection needle or a syringe.

The present invention provides a method of separating adipose tissue-derived stromal vascular fraction, the method including: providing the kit for separating the adipose tissue-derived stromal vascular fraction; injecting a collected adipose tissue and a lipolytic agent into a first separation tube of the kit through an opening portion and then centrifuging; injecting an injection needle or a syringe to the centrifuged bottom hole of the first separation tube to take out a content including stromal vascular fraction from a lower storage portion; washing the stromal vascular fraction by injecting a washing solution and the content including the stromal vascular fraction into a second separation tube of the kit through an opening portion of the second separation tube to mix and then centrifuging; and closing the centrifuged passage portion of the second separation tube with a closing adjustment screw, shaking the second separation tube, and injecting an injection needle or a syringe to a bottom hole of the second separation tube to take out a solution including the stromal vascular fraction.

### Advantageous Effects

According to the present invention having above-mentioned construction, a kit for separating adipose tissue-derived stromal vascular fraction is easy to operate so that even non-specialists can use, and performs washing and suspending processes repeatedly for the stromal vascular fraction in one tube at the same time, thereby minimizing exposure to air, resulting in a low risk of secondary infection caused by external agents. Accordingly, since the survival rate and recovery rate of the stromal vascular fraction with respect to the amount of adipose tissue are increased, less adipose tissue is required and thus the stromal vascular fraction is separated from the adipose tissue efficiently and easily.

### DESCRIPTION OF DRAWINGS

FIG. 1 is a vertical sectional view of a first separation tube according to an embodiment of the present invention;

FIG. 2 is a vertical sectional view of a second separation tube according to the embodiment of the present invention;

FIG. 3 is a vertical sectional view of a second separation tube according to another embodiment;

FIG. 4 is a vertical sectional view showing a passage portion of a second separation tube is closed with a closing adjustment screw;

FIG. 5 is a vertical sectional view showing a passage portion of a second separation tube according to another embodiment is closed with a closing adjustment screw;

FIG. 6 is a vertical sectional view showing an upper portion of a first separation tube having two opening portions and a cap closing the opening portions;

FIG. 7 is a vertical sectional view showing an upper portion of a second separation tube having two opening portions and a cap closing the opening portions;

FIG. 8 is an enlarged vertical sectional view showing a passage portion having a scale of a second separation;

FIG. 9 is an enlarged vertical sectional view showing a closing adjustment screw provided with a pressure control ring including a plurality of holes and a blocking plate opening and closing the holes;

FIG. 10 is a top view showing a pressure control ring and a blocking plate disposed on the pressure control ring;

FIG. 11 is a vertical sectional view showing that a pressure generated when a closing adjustment screw is moved upward presses an upper blocking plate whereby holes of a pressure control ring are blocked;

FIG. 12 is a top view showing that an upper blocking plate and a lower blocking plate cover different holes;

FIG. 13 is a top view showing an upper blocking plate and a lower blocking plate are configured in a separated type rather than an integral type as shown in FIG. 10, and which cover different holes of a pressure control ring;

FIG. 14 is a top view showing an upper surface of an upper storage portion of a first separation tube, in which the upper surface is provided with an air outlet;

FIG. 15 is a cross-sectional view showing an air outlet provided on an upper surface of an upper storage portion of a first separation tube, in which the air outlet may be provided with a membrane having a plurality of fine holes;

FIG. 16 is a top view showing an upper surface of an upper storage portion of a second separation tube, in which the upper surface is provided with an air vent; and

FIG. 17 is a cross-sectional view showing an air vent provided on an upper surface of an upper storage portion of a second separation tube, in which the air vent is provided with an air cleaning filter and a membrane having a plurality of fine holes disposed below the air cleaning filter.

### BEST MODE

Hereinafter, a kit for separating adipose tissue-derived stromal vascular fraction and a method of separating adipose tissue-derived using same according to the present invention will be described with reference to the accompanying drawings. In the following description, it is to be noted that, when the functions of conventional elements and the detailed description of elements related with the present invention may make the gist of the present invention unclear, a detailed description of those elements will be omitted. In addition, it should be understood that the shape and size of the elements shown in the drawings may be exaggeratedly drawn to provide an easily understood description of the structure of the present invention.

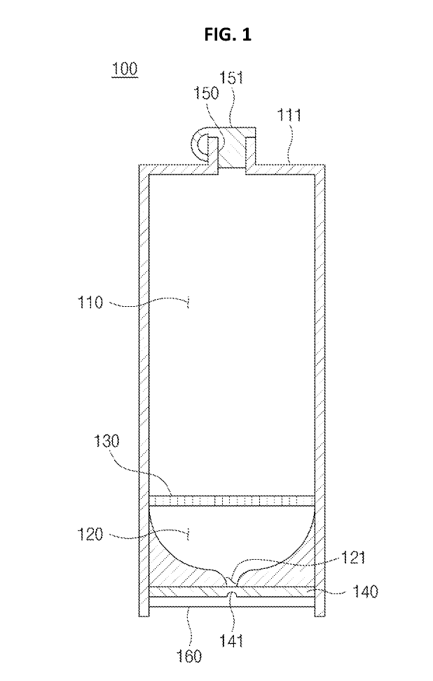

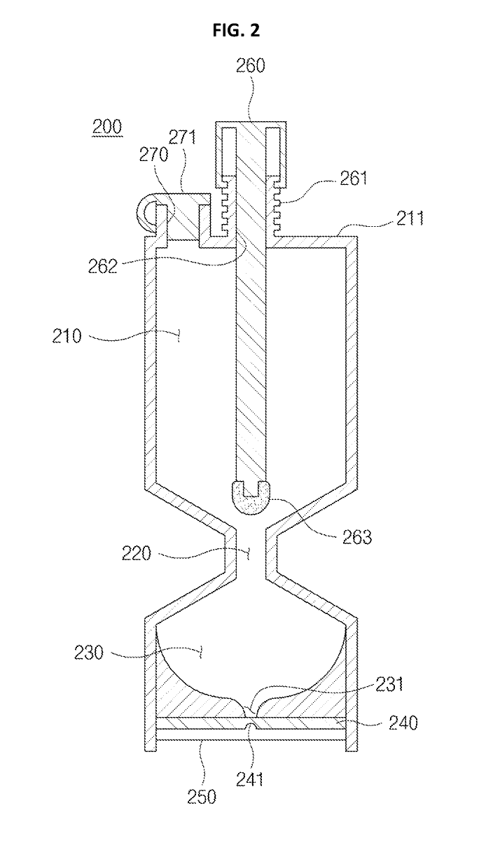

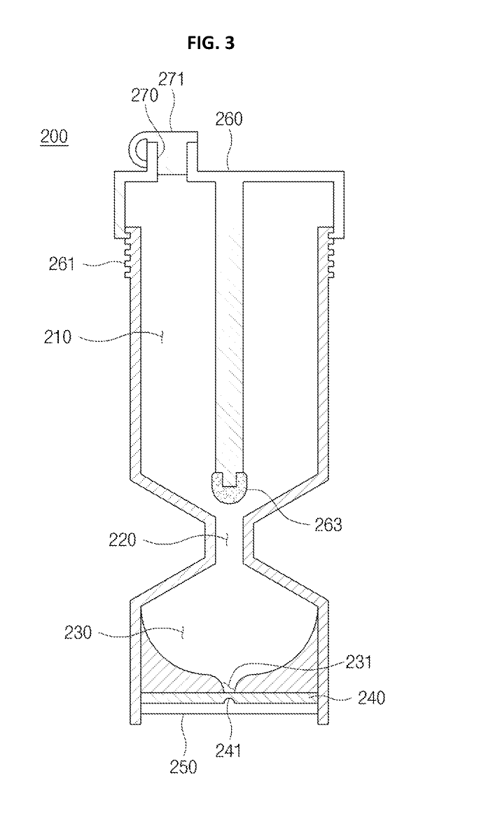

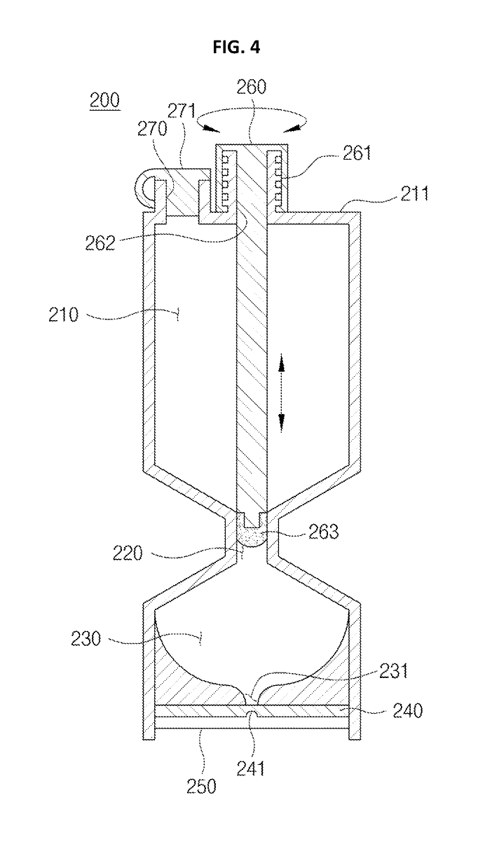

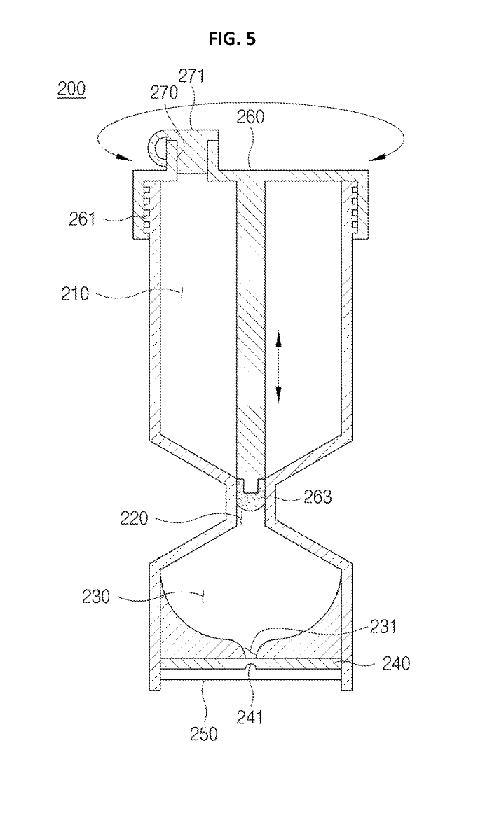

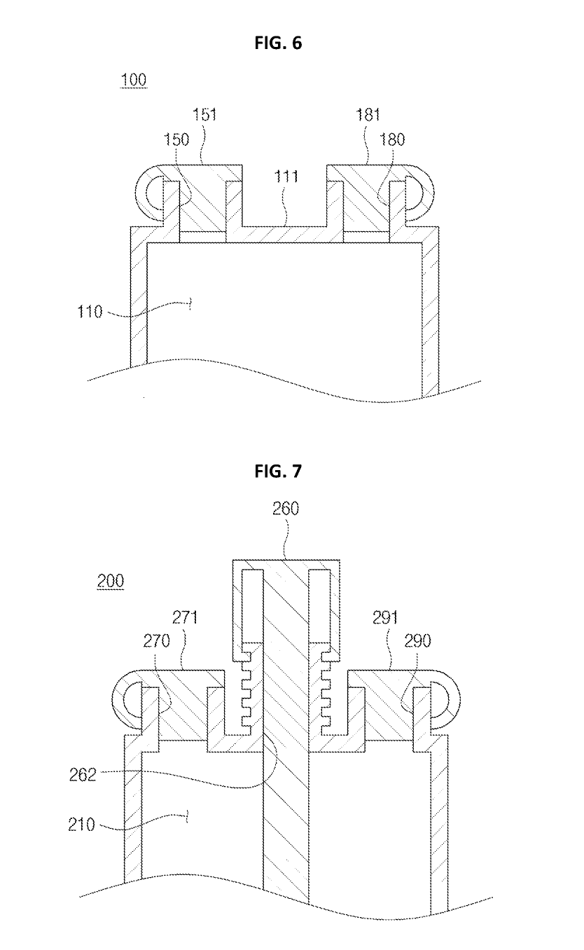

FIG. 1 is a vertical sectional view of a first separation tube 100 included in the present invention. FIGS. 2 and 3 are vertical sectional views of second separation tubes 200 included in the present invention according to respective embodiments. FIGS. 4 and 5 are vertical sectional view each showing passage portion 220 of the second separation tubes 200 of FIGS. 2 and 3 are tightly closed with a closing adjustment screw 260. FIGS. 6 and 7 are vertical sectional views each showing the first separation tube 100 and the second separation tube 200 each having two opening portions.

As shown in FIG. 1, a first separation tube 100 included in a kit for separating adipose tissue-derived stromal vascular fraction according to the present invention includes an upper storage portion 110, a lower storage portion 120 tapering downwards, a filter 130 separating the upper storage portion 110 and the lower storage portion 120 from each other and allowing the stromal vascular fraction to pass therethrough but not the adipose tissue, at least one opening portion 150 for introducing/discharging a material and air, a cap 151 closing the opening portion, a bottom hole 121 disposed at a lower end of the lower storage portion, and a bottom hole blocking film 140 disposed at a lower end of the bottom hole to protect the bottom hole from outside and composed of a material penetrated by an injection needle or a syringe.

FIG. 6 is a vertical sectional view showing the first separation tube having two opening portions 150. When the first separation tube has a plurality of the opening portions 150 and 180, the opening portions may be used for introducing/discharging air and materials used for separating the stromal vascular fraction respectively. A plurality of caps 151 and 181 closing the plurality of opening portions may be installed in various forms, for example, the caps may be configured to open at the same time as the caps connect to each other.

FIG. 7 is a vertical sectional view showing the second separation tube having two opening portions 270. As described in FIG. 6, the opening portions 270 and 290 may be used for introducing/discharging air and materials used for separating the stromal vascular fraction. A plurality of caps 271 and 291 closing the plurality of opening portions may be installed in various forms, for example, the caps may be configured to open at the same time as the caps connect to each other.

The drawings describe that the caps 151, 181, 271, and 291 each closing the opening portions 150, 180, 270, and 290 of the first separation tube 100 and the second separation tube 200 engage on upper ends of the upper storage portions, but it is not limited thereto, and the caps may be separated therefrom. In addition, it is preferable that a material for the caps 151, 181, 271, and 291 has flexibility, thereby closing the opening portion firmly.

The first separation tube 100 is a tube for separating the stromal vascular fraction from collected adipose tissue. When the adipose tissue and lipolytic enzyme are put together into the upper storage portion 110 through the opening portion 150 of the first separation tube and centrifugation is performed, the stromal vascular fraction moves to the lower storage portion 120 except the adipose tissue. Such separation is possible due to the filter 130 that differentiates the upper storage portion 110 and the lower storage portion 120 of the first separation tube because the stromal vascular fraction passes through the filter 130 but not the adipose tissue. The filter 130 may filter out not only the adipose tissue but also other unwanted tissues. In addition, the filter 130 may have a very fine hole, and a diameter of the hole is preferably 40 μm to 100 μm, but not limited thereto.

As shown in FIGS. 2 and 3, the second separation tube 200 included in the kit for separating the adipose tissue-derived stromal vascular fraction according to the present invention to wash and suspend the stromal vascular fraction includes an upper storage portion 210 tapering downwards and provided with a spiral 261 at an upper end thereof, a lower storage portion 230 tapering upwards and downwards, the passage portion 220 connecting between the upper storage portion 210 and the lower storage portion 230, at least one opening portion 270 for introducing/discharging a material and air, the cap 271 closing the opening portion 270, a closing adjustment screw 260 capable of moving vertically, closing the passage portion 220, and engaging with the spiral 261 provided on the upper end of the upper storage portion 210, a bottom hole 231 disposed at a lower end of the lower storage portion, and a bottom hole blocking film 240 composed of a material penetrated by an injection needle or a syringe, disposed at a lower end of the bottom hole 231 to protect the bottom hole 231 from outside.

In the second separation tube, the combination of the closing adjustment screw 260 and the upper storage portion 210 is not limited thereto, but may be implemented as shown in FIGS. 2 and 3.

As shown in FIG. 2, the upper storage portion 210 of the second separation tube may further include a projection 262 at an upper end 211 thereof, and the spiral 261 where the closing adjustment screw 260 engages may be formed inside or outside of the projection.

In addition, as shown in FIG. 3, a head portion of the closing adjustment screw 260 engaged with the spiral 261 provided on the upper end of the upper storage portion of the second separation tube may be configured to function as a cap covering an entire cross-sectional area of the upper storage portion 260. At this point, the opening portion 270 may be disposed at the head portion of the closing adjustment screw 260, but not limited thereto.

As shown in FIGS. 4 and 5, the passage portion 220 may be closed by turning the closing adjustment screw 260.

The second separation tube 200 is a tube for washing and suspending the stromal vascular fraction separated by the first separation tube 100.

The stromal vascular fraction separated by the first separation tube 100 needs to be washed due to foreign substances such as lipolytic enzyme. In some cases, washing may be repeated several times, and it is possible that the stromal vascular fraction floats during an exchange of a washing solution and may be washed away. However, the second separation tube 200 according to the present invention tightly closes the passage portion 220 with the closing adjustment screw 260 to prevent the stromal vascular fraction in the lower storage portion 230 from discharging, therefore the stromal vascular fraction is not discharged during additional washing processes.

In addition, in a suspension process, the stromal vascular fraction is injected to the second separation tube 200 and the passage portion 220 is closed with the closing adjustment screw 260. Then, after a deposited stromal vascular fraction in the lower storage portion 230 floats by shaking the second separation tube 200, suspension with a high concentration of the stromal vascular fraction can be obtained since the stromal vascular fraction exists in the lower storage portion 230.

A silicone cap 263 may be provided at an end of the closing adjustment screw 260 to improve sealing.

Each of the bottom hole blocking film 140 of the first separation tube 100 and the bottom hole blocking film 240 of second separation tube 200 may be provided with respective grooves 141 and 241 at each lower end surface, the groove 141 and 241 each indicating a position of the bottom hole 121 and 231 disposed at the lower end of the lower storage portion 120 and 230 respectively at each lower end surface. The grooves 141 and 241 suggest where to position a syringe properly by indicating the respective positions of the bottom holes 121 and 231.

In addition, each of the first separation tube 100 and the second separation tube 200 may further include respective sealing membranes 160 and 250 at a lower portion of the blocking films of the lower portion 140 and 240, respectively, and the sealing membranes 160 and 250 is preferably composed of a material penetrated by an injection needle. The stromal vascular fraction is collected sanitarily due to the further included sealing membranes 160 and 250.

Furthermore, a cylindrical shape may be preferable for each overall shape of the first separation tube 100 and the second separation tube 200, but not limited thereto. When the first separation tube 100 and the second separation tube 200 have cylindrical shapes, it is possible to stand on the floor, thereby improving convenience. In addition, the upper storage portions 110 and 210, the lower storage portion 120 and 230, and the passage portion 220 of the second separation tube are preferably made of a transparent material to enable observation from outside.

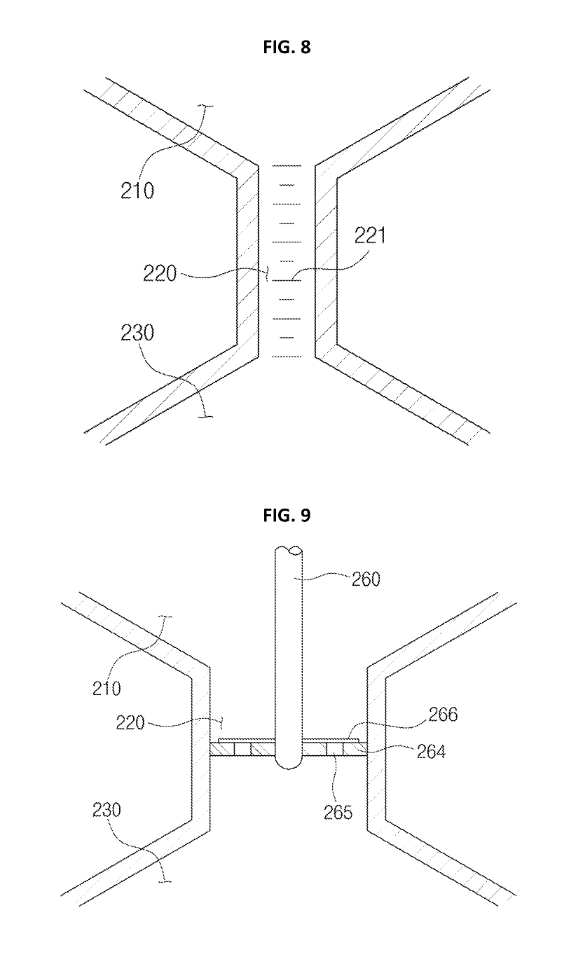

FIG. 8 is an enlarged view showing the passage portion 220 having a scale 221 of the second separation tube.

The purposes of separating the stromal vascular fraction are various, such as storage and transplantation. For each purpose, a suitable volume of the stromal vascular fraction suspension is different. Most of the techniques currently involve the extraction process of the stromal vascular fraction and then suspension process to achieve a desired volume. During the process, the stromal vascular fraction may be exposed to air and become contaminated or necrotic. Therefore, those skilled in the art keenly need a kit capable of adjusting a volume thereof.

A function adjusting a volume of a solution including the stromal vascular fraction may be further included, the function increasing or decreasing a length or a cross-sectional area of the passage portion 220 to adjust a volume between a lower end of the closing adjustment screw 260 and a lower end of the passage portion 220. In such case, an accurate volume adjustment becomes possible by indicating the scale 221 on the passage portion 220. If the length or the cross-sectional area of the passage portion 220 varies, it is preferable that the closing adjustment screw 260 is extended in length to reach the bottom of the passage portion 220 or is changed in thickness to close the passage portion 220.

The closing adjustment screw 260 moves vertically inside the passage portion 220. At this point, if a pressure is not adjusted, the movement of the closing adjustment screw 260 may not be smooth. When the closing adjustment screw 260 decreases the volume of the solution including the stromal vascular fraction, the pressure of the solution may be increased whereby the stromal vascular fraction may be stressed and necrotized. Therefore, it is possible to add a configuration for adjusting the pressure. Hereinafter, a specific embodiment of the configuration for adjusting the pressure will be described with reference to FIGS. 9 to 13.

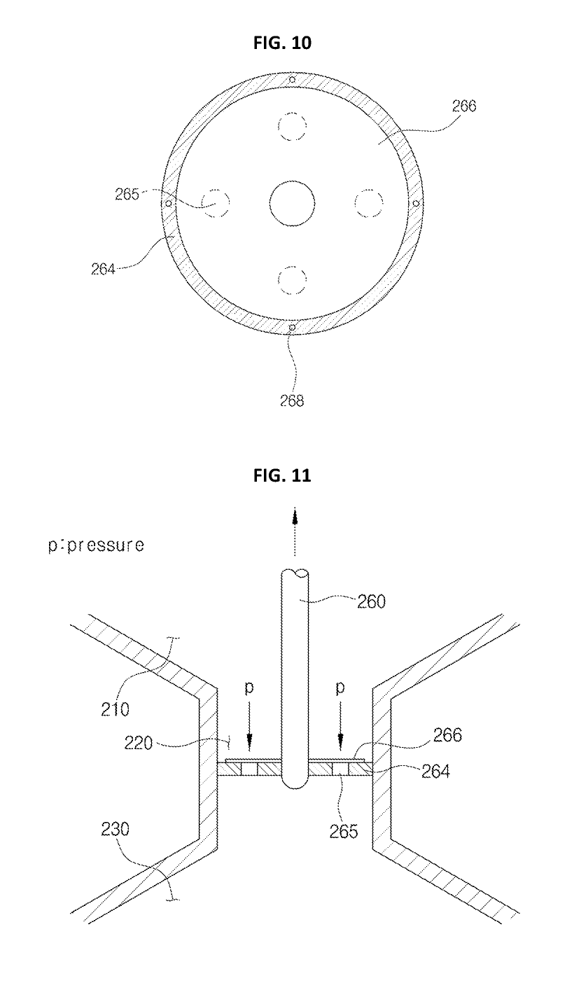

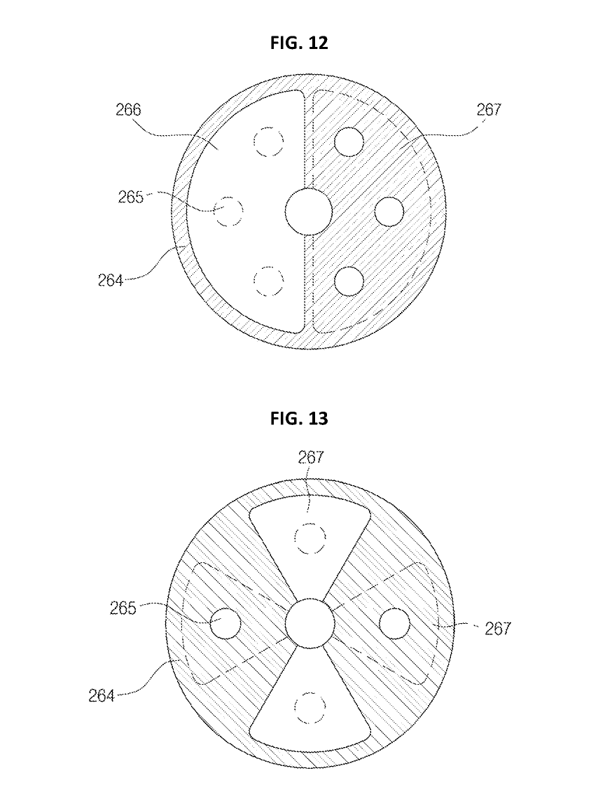

Each of FIGS. 9 to 13 is a diagram showing a pressure control ring 264 controlling a pressure to smooth the vertical movement of the closing adjustment screw 260. FIG. 9 is an enlarged vertical sectional view showing the closing adjustment screw 260 provided with the pressure control ring 264 including a plurality of holes 265 and a blocking plate 266 opening and closing the holes 265. FIG. 10 is a top view showing the pressure control ring 264 and the blocking plate 266 disposed on the pressure control ring. FIG. 11 is a vertical sectional view showing that a pressure generated when the closing adjustment screw 260 is moved upward presses an upper blocking plate 266 whereby the holes 265 of the pressure control ring 264 are blocked. In addition, FIG. 12 is a top view showing that the upper blocking plate 266 and a lower blocking plate 267 cover different holes 265 of the pressure control ring 264. FIG. 13 is a top view showing that the upper blocking plate 266 and the lower blocking plate 267 are configured as a separated type rather than an integral type as shown in FIG. 10, and which cover different holes of the pressure control ring.

As shown in FIG. 9, a configuration for controlling the pressure is implemented by the pressure control ring 264 including the plurality of holes 265. Since movements of air and liquid through the plurality of holes 265 are free, the movement of the closing adjustment screw 260 becomes smooth.

In addition, the pressure control ring 264 may be provided with the blocking plate 266 opening and closing the holes 265 at an upper end thereof or both upper and lower ends thereof. A reason for the presence of the blocking plate 266 is to prevent the material of the upper storage portion from being introduced and discharged with the material of the lower storage portion, which is required to be discharged to outside. Therefore, it is preferable to provide the blocking plate 266 to extract a specific volume of the material.

The blocking plate 266 may be disposed only on the pressure control ring 264 as shown in FIGS. 9 and 10. On the other hand, the blocking plate 266 may be disposed on the pressure control ring 264 and below the pressure control ring 264 as shown in FIGS. 12 and 13, and at this point, it is preferable the upper blocking plate 266 and the lower blocking plate 267 open and close the plurality of holes 265 without overlapping each other. More specifically, the fact that the upper blocking plate 266 and the lower blocking plate 267 can open and close the plurality of holes 265 without overlapping each other means that the upper blocking plate 266 and the lower blocking plate 267 open and close different holes.

When the blocking plate 266 is disposed only on the volume control ring 264 as embodiments shown in FIGS. 9 and 10, the closing adjustment screw 260 moves downward smoothly. However, when the closing adjustment screw 260 moves upward as shown in FIG. 11, since the holes 265 are blocked due to the pressure pressing the blocking plate 266, it is not possible for liquid and air exiting on an upper portion of the volume control ring 260 to pass through the pressure control ring 260 to move downward. Thus, it is hard to move the closing adjustment screw 260 whereby it is hard to increase the volume. Therefore, as shown in FIG. 10, the volume control ring 260 existing only on the upper blocking plate 266 may further include at least one fine hole 268, and gas is capable of passing through the fine hole 268 but not liquid. Since only gas is capable of passing the fine hole 268, it is preferable to move the closing adjustment screw 260 upward after emptying the liquid existing in the upper portion of the pressure control ring 264.

As shown in FIGS. 12 and 13, in case of the upper blocking plate 266 and the lower blocking plate 267 are provided, the lower blocking plate 267 is opened when the closing adjustment screw 260 moves upward, and the upper blocking plate 266 is opened when the closing adjustment screw 260 moves downward, whereby the closing adjustment screw 260 always moves smoothly.

The blocking plate 266 is preferably composed of a flexible material to open and close easily according to a flow of the liquid or gas, and the flexible material may be silicone, rubber, or plastic, but preferably silicone.

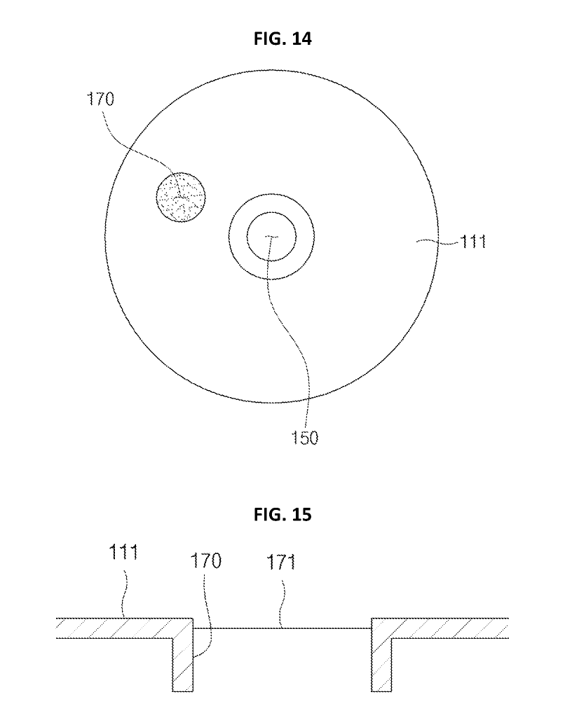

FIG. 14 is a top view showing an upper surface 111 of the upper storage portion of the first separation tube 100, in which the upper surface 111 is provided with an air outlet 170. FIG. 15 is a cross-sectional view showing the air outlet 170 provided on the upper surface of the upper storage portion of the first separation tube. The air outlet 170 may be provided with a membrane 171 having a plurality of fine holes.

When the adipose tissue is introduced into the first separation tube 100, a corresponding volume of air inside the first separation tube is required to be discharged to outside. Therefore, the upper surface 111 of the upper storage portion of the first separation tube 100 may be provided with the air outlet to discharge air inside the first separation tube during introduction of the adipose tissue. The air outlet may be formed in a simple hole 170 as shown in FIG. 14, or provided with the membrane 171 having the plurality of fine holes disposed on the hole 170 as shown in FIG. 15.

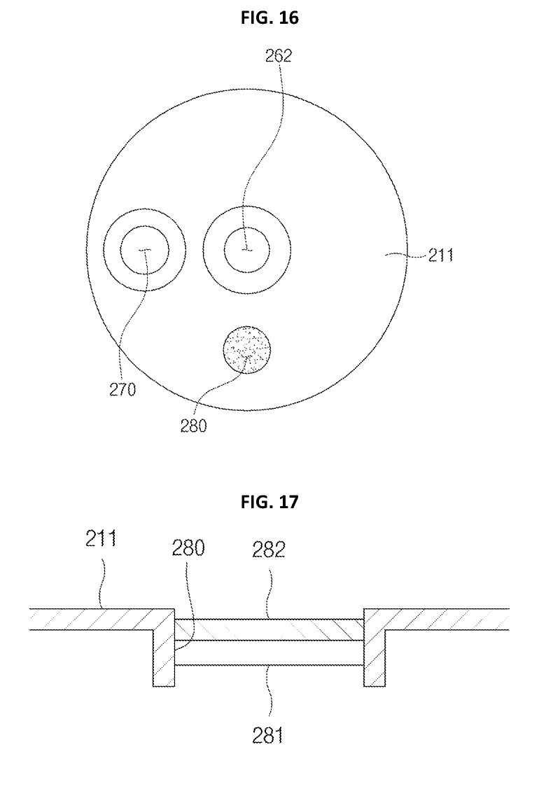

FIG. 16 is a top view showing an upper surface of the upper storage portion of the second separation tube, in which the upper surface is provided with an air vent 280. FIG. 17 is a cross-sectional view showing the air vent 280 provided on the upper surface of the upper storage portion of the second separation tube. The air vent 280 is provided with an air cleaning filter 282 and a membrane 281 having a plurality of fine holes disposed below the air cleaning filter 282.

When using the second separation tube 200, the content may be frequently discharged from the second separation tube, for example, by changing a washing solution several times or injecting the suspension again after removing the washing solution. At this point, when the content is injected into or discharged from the second separation tube 200, a corresponding volume of inside air is discharged, or outside air is introduced into the second separation tube. Therefore, the upper surface 211 of the upper storage portion of the second separation tube 200 may be provided with the air vent at an upper part thereof to introduce and discharge air. The air vent may be formed in a simple hole 280 as shown in FIG. 16, or provided with the membrane 281 having the plurality of fine holes as shown in FIG. 17.

When outside air is introduced into the second separation tube 200, the air cleaning filter 282 purifying outside air may be provided inside the air vent 280 for introducing and discharging air or may be provided inside the opening portion 290 since the second separation tube may be contaminated by the outside air. As shown in FIG. 17, when the air cleaning filter 282 is provided with the membrane 281 having the plurality of fine holes, the air cleaning filter 282 is preferably disposed above the membrane 281.

It is preferable that the membranes 171 and 281 provided on the air outlet 170 and the air vent 280 and having fine holes allow only air to pass therethrough, and the contents inside the first separation tube 100 and the second separation tube 200 do not leak.

The stromal vascular fraction is capable of being separated from the adipose tissue by using the kit according to the present invention. A separating method is implemented by following steps.

The method includes providing the kit for separating the adipose tissue-derived stromal vascular fraction according to the present invention, injecting a collected adipose tissue and a lipolytic agent into the first separation tube 100 of the kit through the opening portion 150 and then centrifuging, injecting an injection needle or a syringe to the centrifuged bottom hole 121 of the first separation tube to take out a content including the stromal vascular fraction from the lower storage portion 120, washing the stromal vascular fraction by injecting the content including the stromal vascular fraction and a washing solution into the second separation tube 200 of the kit through the first opening portion 270 to mix and then centrifuging, and closing the centrifuged passage portion 220 of the second separation tube with the closing adjustment screw 260, shaking the second separation tube 200, and injecting an injection needle or a syringe to the bottom hole 231 of the second separation tube to take out a solution including the stromal vascular fraction.

The lipolytic agent may include lipolytic enzyme, fibrous protease, and the like. The lipolytic enzyme may be tumescent, lipase, phospholipase, or esterase. The fibrous protease may be collagenase, ACCUTASE, peptidase, endopeptidase, or exopeptidase.

After the washing the stromal vascular fraction, the method may further include closing the centrifuged passage portion 220 of the second separation tube with the closing adjustment screw 260 and taking out a content in the upper storage portion 210 of the second separation tube to empty the upper storage portion, loosening the closing adjustment screw 260 to open the passage portion 220, introducing a suspension into the empty upper storage portion 210 through the first opening portion 270, and centrifuging the second separation tube, and closing the centrifuged passage portion 220 of the second separation tube with the closing adjustment screw 260, shaking the second separation tube 200, and injecting an injection needle or a syringe to the bottom hole 231 of the second separation tube to take out a stromal vascular fraction suspension formed by mixing the suspension and the stromal vascular fraction together, thus the stromal vascular fraction is obtained in a form of a suspension.

Hereinbelow, the present invention will be described in detail with reference to an example. The example is only for illustrating the present invention more specifically, and it will be apparent to those skilled in the art that the scope of the present invention is not limited by the example.

### EXAMPLE

### Extraction of Stromal Vascular Fraction from Adipose Tissue

1. 20 mL of adipose tissue and 20 mL of collagenase are introduced into a first separation tube, and the adipose tissue and a collagenase solution are put into a shaking incubator at 37° C. for 40 minutes to react each other.

2. Centrifugation is performed in 1500 rpm for five minutes. After taking out about 10 ml of a solution, which includes stromal vascular fraction and is deposited in a lower storage portion of the first separation tube, by a syringe, the solution is introduced into a second separation tube.

3. The stromal vascular fraction is washed by introducing 60 mL of a washing solution and centrifugation is performed in 1500 rpm for five minutes.

4. A passage portion is closed with a closing adjustment screw after the centrifugation is complete. An upper layer of the washing solution is discharged and replaced with a clean washing solution. The closing adjustment screw is raised to mix a lower layer of the suspension and the clean washing solution together.

5. Centrifugation is performed in 1500 rpm for five minutes.

6. The passage portion is closed with the closing adjustment screw after the centrifugation is complete. 5 ml to 10 ml (final volume control) of the solution, which is in a lower storage portion of the second separation tube and includes the stromal vascular fraction, is collected by a syringe.

The number of stromal vascular fraction cells included in the collected solution was 2.5×10⁷ cell, thus it is known that the number of separated stromal vascular fraction per 1 mL of adipose tissue is 1.25×10⁶ cells. In addition, a survival rate of the stromal vascular fraction was measured by using an automated cell counting device (LUNA Stem of Logos or CountessII automated cell counter of Life technologies), and a result was 96.5%. Table 1 below shows performances of conventional machines for extracting the stromal vascular fraction from the adipose tissue (published at Technology Seminar for revitalization of stem cell related industry, conducted on Mar. 18, 2011 (organizer: Gyeongi Institute of Science & Technology Promotion-Industry Foundation GyeongGi Bio Center)).

TABLE 1

| | The number of stromal vascular fraction |
|---|---|
| | cells extracted from 1 mL of adipose |
| | tissue (×10⁶) |
| Multi-station | 0.304 ± 0.16 |
| (Spectronix) | |
| CHA-Station (PNC | 0.361 ± 0.19 |
| International) | |
| Cytori's Celution | 0.290 ± 0.10 |
| (Cytori) | |
| Maxstem YC-100 | 1.09 |
| (Biomaster) | |
| TGI-1000TM (TGI) | 1.180 ± 0.45 |

From Table 1, it is known that the kit of the present invention has an excellent recovery rate of the stromal vascular fraction.

What is claimed is:

1. A kit for separating adipose tissue-derived stromal vascular fraction, the kit comprising:
(a) a first separation tube for separating stromal vascular fraction from adipose tissue, the first separation tube including an upper storage portion, a lower storage portion tapering downwards, a filter separating the upper storage portion and the lower storage portion from each other and allowing the stromal vascular fraction to pass therethrough but not the adipose tissue, at least one opening portion for introducing/discharging a material and air, a cap closing the opening portion, a bottom hole disposed at a lower end of the lower storage portion, and a bottom hole blocking film disposed at a lower end of the bottom hole to protect the bottom hole from outside and composed of a material penetrated by an injection needle or a syringe; and
(b) a second separation tube for washing and suspending the stromal vascular fraction, the second separation tube including an upper storage portion tapering downwards and provided with a spiral at an upper end thereof, a lower storage portion tapering upwards and downwards, a passage portion connecting between the upper storage portion and the lower storage portion, at least one opening portion for introducing/discharging a material and air, a cap closing the opening portion, a closing adjustment screw capable of moving vertically, closing the passage portion, and engaging with the spiral provided on an upper end of the upper storage portion, a bottom hole disposed at a lower end of the lower storage portion, and a bottom hole blocking film composed of a material penetrated by an injection needle or a syringe, disposed at a lower end of the bottom hole to protect the bottom hole from outside.

2. The kit of claim 1, wherein a head portion of the closing adjustment screw engaged with the spiral provided on the upper end of the upper storage portion of the second separation tube is a cap for the upper storage portion.

3. The kit of claim 1, wherein the upper storage portion of the second separation tube further includes a projection at an upper end thereof, and the spiral where the closing adjustment screw engages is formed inside or outside of the projection.

4. The kit of claim 1, wherein a silicone cap is provided at an end of the closing adjustment screw of the second separation tube to close the passage portion tightly.

5. The kit of claim 1, wherein the passage portion of the second separation tube has a scale to adjust a volume of contents in the lower storage portion.

6. The kit of claim 1, wherein the closing adjustment screw of the second separation tube is provided with a pressure control ring at an end thereof, the pressure control ring including a plurality of holes.

7. The kit of claim 6, wherein the pressure control ring is provided with a blocking plate at an upper end thereof or a blocking plate at each of an upper end and a lower end thereof, the blocking plate opening and closing the plurality of holes.

8. The kit of claim 7, wherein an upper blocking plate and a lower blocking plate open and close different holes when the blocking plates are provided on the upper and lower ends of the pressure control ring.

9. The kit of claim 1, wherein each bottom hole blocking film of the first separation tube and the second separation tube is provided with a groove at a lower end surface, the groove indicating a position of the bottom hole disposed at a lower end of the lower storage.

10. The kit of claim 1, wherein each of the first separation tube and the second separation tube further includes a sealing membrane at a lower portion of the blocking film of the lower storage portion, and the sealing membrane is composed of a material penetrated by an injection needle or a syringe.

11. A method of separating adipose tissue-derived stromal vascular fraction, comprising:
providing the kit for separating the adipose tissue-derived stromal vascular fraction of claim 1;
injecting a collected adipose tissue and a lipolytic agent into a first separation tube of the kit through an opening portion and then centrifuging;
injecting an injection needle or a syringe to the centrifuged bottom hole of the first separation tube to take out a content including stromal vascular fraction from a lower storage portion;
washing the stromal vascular fraction by injecting a washing solution and the content including the stromal vascular fraction into a second separation tube of the kit through an opening portion of the second separation tube to mix and then centrifuging; and
closing the centrifuged passage portion of the second separation tube with a closing adjustment screw, shaking the second separation tube, and injecting an injection needle or a syringe to a bottom hole of the second separation tube to take out a solution including the stromal vascular fraction.
